(19) United States
(12) Patent Application Publication
(10) Pub. No.: US 2026/0062181 A1
(43) Pub. Date: Mar. 5, 2026

(54) MULTISTAGE VALVE SYSTEM FOR MULTIDOSE BOTTLES

(71) Applicant: Alcon Inc., Fribourg (CH)

(72) Inventor: Jeffrey Thomas Mitchell, Mansfield, TX (US)

(21) Appl. No.: 19/317,222

(22) Filed: Sep. 3, 2025

Related U.S. Application Data
(60) Provisional application No. 63/690,986, filed on Sep. 5, 2024.

(51) Int. Cl. B65D 47/20; A61F 9/00; A61J 1/14; B01D 46/00; B65D 47/12; B65D 47/32

(52) U.S. Cl. CPC B65D 47/2075; A61F 9/0008; A61J 1/145; B01D 46/0028; B65D 47/121; B65D 47/32; B01D 2279/65

(57) ABSTRACT

In some embodiments, an apparatus for dispensing fluids includes a manifold, a fluid path at least partially disposed in the manifold and having an inlet and an outlet, and a multistage valve at least partially disposed in the manifold and relative to the fluid path. The multistage valve includes a core positioned in the fluid path between the inlet and the outlet, a seal positioned around the fluid path between the inlet and the outlet, and a biasing device oriented to cause a first force to be exerted on the core. The first force biases the core in a first direction against the seal to close the fluid path. The multistage valve further includes a plurality of diaphragms positioned adjacent to the fluid path, wherein the plurality of diaphragms acts upon the core in response to fluid pressure on an inlet side of the fluid path.

### INTRODUCTION

Many pathologies of the eye are treated by direct application of fluids to the eye, such as liquid eye drops. For example, conjunctivitis is treated by directly applying eye drops containing antibiotics. Dry eyes and glaucoma are also treated using eye drops. In general, eye drops and other treatment fluids can be used as lubricants for relieving discomfort and dryness of the eyes, as well as delivery vehicles for therapeutic substances.

Eye drops, as well as other treatment fluids, are typically stored in a flexible container with a built-in dispensing device. To apply a treatment fluid to a patient's eye, the flexible container is manually compressed by a user to dispense the treatment fluid through the dispensing device. However, there is a risk of microbial ingress to the container.

### SUMMARY

Embodiments of the present disclosure provide a device, or apparatus, for dispensing fluids (including liquids, gels, solutions, emulsions, and suspensions) from flexible containers, such as multidose preservative free (MDPF) bottles, while providing improved fluid flow control and reduction/prevention of microbial ingress into the containers.

In some embodiments, an apparatus for dispensing fluids is provided, the apparatus including: a manifold; a fluid path at least partially disposed in the manifold, the fluid path including an inlet and an outlet; and a multistage valve at least partially disposed in the manifold and relative to the fluid path, the multistage valve including: a core positioned in the fluid path between the inlet and the outlet; a seal positioned around the fluid path between the inlet and the outlet, the core nested with the seal; a biasing device oriented to cause a first force to be exerted on the core, the first force biasing the core in a first direction against the seal to close the fluid path; and a plurality of diaphragms positioned adjacent to the fluid path, wherein the plurality of diaphragms is configured to act upon the core in response to fluid pressure on an inlet side of the fluid path exerting a second force against the plurality of diaphragms to at least partially unnest the core from the seal.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 is a schematic cross-sectional side view of a device for dispensing fluids, according to some embodiments of the present disclosure.

FIG. 2 is a schematic cross-sectional side view of another device for dispensing fluids, according to some embodiments of the present disclosure.

FIG. 3A is a partial schematic cross-sectional top view of a component of the device of FIG. 1 or FIG. 2, according to some embodiments of the present disclosure.

FIG. 3B is a partial schematic cross-sectional side view of a portion of the device of FIG. 1, according to some embodiments of the present disclosure.

FIG. 4 is a schematic cross-sectional side view of the device of FIG. 1 with a cap disposed thereon, according to some embodiments of the present disclosure.

FIG. 5A is a schematic cross-sectional side view of the device of FIG. 1 when attached to a flexible container, according to some embodiments of the present disclosure.

FIG. 5B is a schematic cross-sectional side view of the device of FIG. 1 during dispensing, according to some embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments of the present disclosure provide devices for dispensing fluids (including liquids, gels, solutions, emulsions, and suspensions) from flexible containers, while providing improved fluid flow control and reduction or even prevention of microbial ingress into the containers. Examples of flexible containers include eye drop dispensers, droptainers, drop bottles, squeeze bottles, dropping bottles, and squeeze tubes. To actuate the dispensing of fluids contained within a flexible container, portions of a body of the flexible container may be compressed or squeezed by a user, pump, or other actuation mechanism (including electronically controlled mechanisms). This compression increases the pressure within the flexible container and/or compresses the fluids therein, thereby causing the fluids to be expelled through an opening, or outlet, of the flexible container. The devices of the present disclosure can be coupled to the opening of the flexible container to provide improved fluid flow control and reduced/prevented microbial ingress into the container.

In some embodiments, the devices may be utilized for dispensing of ophthalmic fluids, including lubricating eye drops, medicated/prescription eye drops, ophthalmic gels, ophthalmic ointments, ophthalmic stains, and other preservative free ocular solutions.

In some embodiments, the devices include a multistage valve mechanism that reduces, and in some examples, prevents microbial ingress when dispensing fluids from the aforementioned flexible containers, while still allowing passive ingress of air. The valve mechanism also facilitates the reduction of pressure for actuating the valve mechanism by a user, regardless of the viscosity of the solution being dispensed. Accordingly, embodiments of the present disclosure relate to a valve mechanism that effectively resists microbial contamination, allows passive air entry, and reduces the force(s) for operating and/or controlling the speed of fluid flow, thereby facilitating improved fluid preservation and flow control.

Examples will now be described relative to the Drawings.

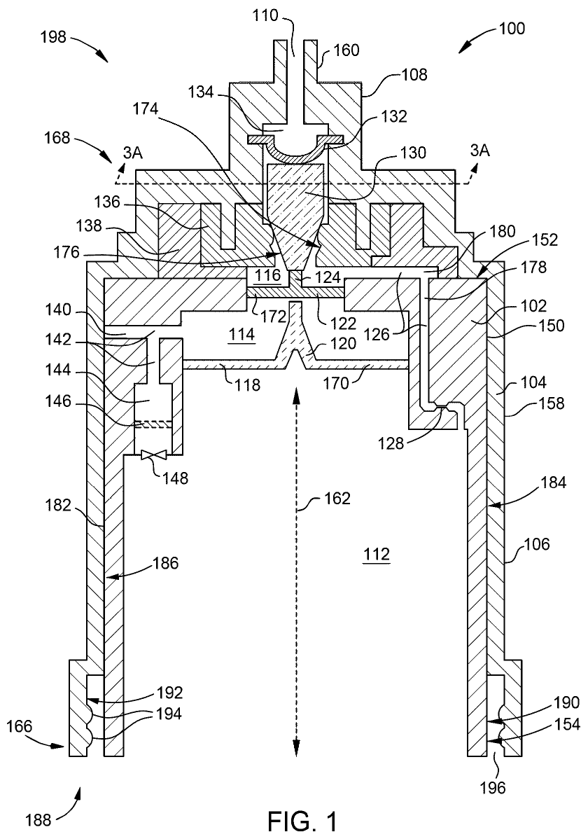

FIG. 1 is a schematic cross-sectional side view of an example dispensing device 100 for dispensing fluids, according to some embodiments of the present disclosure.

In some examples, the dispensing device 100 can be attached to an opening of a flexible container for dispensing ophthalmic fluids. Examples of suitable flexible containers for dispensing ophthalmic fluids may include eye drop dispensers, droptainers, drop bottles, squeeze bottles, dropping bottles, and squeeze tubes. Examples of ophthalmic fluids include lubricating eye drops, medicated/prescription eye drops, ophthalmic gels, ophthalmic ointments, ophthalmic stains, and other preservative free ocular solutions.

In the description herein, “proximal” with reference to the dispensing device 100 or components thereof shall be understood as the end including, or nearest, a base 188 opposite of a dispensing end 198 of the dispensing device 100. “Distal” with reference to the dispensing device 100 or components thereof shall be understood as the end from which, or the end nearest to, the dispensing end 198 where fluid contained in the dispensing device 100 is dispensed.

As shown in FIG. 1, the dispensing device 100 includes a manifold 102 and a fairing 104. In the illustrated embodiment, the manifold 102 includes a body 150 having a generally tubular or cylindrical shape extending from a distal end 152 to a proximal end 154. An axis 162 of the tubular or cylindrical shape of body 150 is shown in FIG. 1 for reference. It should be appreciated that the shape described relative to the manifold 102 is merely illustrative. In various embodiments, the manifold 102 can have any suitable geometry, shape, and/or size including, for example, tapered shapes, ovaloid shapes, and/or the like.

The body 150 of the manifold 102 includes an outer surface 184. In some embodiments, at least a portion of the outer surface 184 is configured to interface with, or couple to, an inner surface 186 of the fairing 104. Accordingly, at least a portion of the outer surface 184 may have a profile complementary to a profile of the inner surface 186 to facilitate a press fit, or snap fit, between the manifold 102 and the fairing 104. In some embodiments, a portion 190 of the outer surface 184 at the proximal end 154 is configured to interface with, or couple to, an inner surface of a dispensing end of a flexible container. For example, the portion 190 can be shaped and/or sized to form a compression fit, or interference fit, with the inner surface of the flexible container when the dispensing device 100 is attached to the flexible container.

The fairing 104 includes a body 158 having a proximal segment 106 at a proximal end 166 of the fairing 104 and a distal segment 108 at a distal end 168 of the fairing 104. The proximal segment 106 is configured to be at least partially disposed around the manifold 102 (for example, slid over the manifold 102, while the distal segment 108 is configured to hold various fluid sealing components of the dispensing device 100. As noted above, the fairing 104 and the manifold 102 may be press fit, or snap fit, together. In some embodiments, the distal segment 108 of the fairing 104 forms, or is coupled to, a nozzle 160 for dispensing of fluids from the dispensing device 100. The distal end 168 of the fairing 104 and the distal end 152 of the manifold 102 may be collectively referred to as the dispensing end 198 of the dispensing device 100, while the proximal end 166 of the fairing 104 and the proximal end 154 of the manifold 102 may be collectively referred to as the base 188 of the dispensing device 100.

Generally, the proximal segment 106 of the body 158 has an overall shape similar to that of the manifold 102, but with larger dimensions. In some embodiments, however, the proximal segment 106 may have a different shape relative to the manifold 102. The distal segment 108 and/or nozzle 160, meanwhile, may have any suitable shape for dispensing fluids. For example, as shown in FIG. 1, the distal segment 108 and/or nozzle 160 may have a stepped shape in some embodiments, while in other embodiments, the distal segment 108 and/or nozzle 160 may have a generally tapered shape. It should be appreciated that the shape shown relative to the fairing 104 is merely illustrative. In various embodiments, the body 158 of the fairing 104 can have any suitable geometry, shape, and/or size including, for example, tapered shapes, ovaloid shapes, and/or the like.

The body 158 of the fairing 104 includes the inner surface 186 at both the proximal segment 106 and the distal segment 108. In some embodiments, at least a portion of the inner surface 186 has a profile complementary to the profile of the outer surface 184 of the manifold 102 to facilitate coupling between the fairing 104 and the manifold 102. In some embodiments, a portion 192 of the inner surface 186 at the proximal end 166 is configured to interface with, or couple to, a dispensing end of a flexible container. For example, the portion 192 can be shaped and/or sized to snap fit or press fit with an outer surface of a dispensing end of the flexible container when the dispensing device 100 is attached to the flexible container. In some embodiments, the portion 192 includes one or more features 194 formed thereon, such as one or more threads, that are configured to mate with corresponding or complementary feature(s) on the outer surface of the flexible container. In some embodiments, the one or more features 194 are alternatively or additionally disposed on the portion 190 of the outer surface 184 of the manifold 102.

In some embodiments, the manifold 102 and the fairing 104 are shaped such that an annular gap 196 is formed between the portion 190 of the outer surface 184 and the portion 192 of the inner surface 186 to facilitate accommodation of a dispensing end of a flexible container when the dispensing device 100 is attached to the flexible container. In some embodiments, the proximal end 166 of the fairing 104 may be flared outward to form the annular gap 196 between the portion 190 and the portion 192, as shown in FIG. 1. In some embodiments, the proximal end 154 of the manifold may alternatively or additionally be flared inward to form the annular gap 196.

In some embodiments, portions of the manifold 102 and/or the fairing 104 are formed of a relatively flexible, or pliant, material. For example, the proximal end 154 and/or the proximal end 166 may be formed of flexible material(s) to facilitate attachment of the base 188 of the dispensing device 100 to a flexible container. Examples of suitable flexible materials include a polypropylene (PP), a low-density polyethylene (LDPE), combinations thereof, and other types and/or grades of polymeric materials. In some embodiments, portions of the manifold 102 and/or the fairing 104 are formed of a rigid, or stiff, material to facilitate greater structural integrity. For example, in some embodiments, the nozzle 160 and/or distal segment 108 of the fairing 104 may be made of a rigid material. Examples of suitable rigid materials include high-density polyethylene (HDPE), polypropylene (PP), cyclic olefin copolymer (COC), cyclic olefin polymer (COP), polyetherimide (PEI), and combinations thereof, and other types and/or grades of polymeric materials.

A first chamber 112 is formed within the manifold 102 adjacent to the proximal end 154 thereof. The first chamber 112 is configured to fluidly couple with an internal volume of a flexible container and is arranged and sized to receive fluids from the flexible container during dispensing. Accordingly, the first chamber 112 forms the starting point of a fluid path within the dispensing device 100 extending from the first chamber 112 to the nozzle 160. In some embodiments, the first chamber 112 is at least partially defined by one or more inner surfaces, or walls, of the manifold 102. At least a portion of the first chamber 112 is defined by a first diaphragm 118 that is disposed at a distal end of the first chamber 112.

The manifold 102 further includes a second chamber 114 disposed therein and adjacent to the distal end 152. The second chamber 114 is arranged between the first diaphragm 118 at a proximal end of the second chamber 114 and a second diaphragm 122 at a distal end of the second chamber 114. The second chamber 114 provides a volume, or space, through which the first diaphragm 118 may deflect during use. In some embodiments, the second chamber 114 is at least partially defined by one or more inner surfaces of the manifold 102, similar to the first chamber 112.

In some embodiments, the second diaphragm 122 also partially defines a third chamber 116 disposed between the distal end 152 of the manifold 102 and the fairing 104. As shown in FIG. 1, the third chamber 116 is further defined by a valve seal 136, a valve seal seat 138, a valve core 130, and the manifold 102. During dispensing, fluids are flowed to the third chamber 116 from the first chamber 112, prior to being flowed past the valve core 130 and expelled through the nozzle 160. In some examples herein, the first diaphragm 118, the second diaphragm 122, the valve core 130, the valve seal 136, and/or a biasing device 132 may be collectively referred to as “the valve.”

A flow-through passage 126 extends between the first chamber 112 and the third chamber 116 to form at least a portion of a fluid path extending between the first chamber 112 and the nozzle 160. Generally, the flow-through passage 126 comprises one or more channels, or conduits, through which fluids can be flowed from the first chamber to the third chamber 116 during dispensing. In the embodiment of FIG. 1, the flow-through passage 126 includes a first channel 178 extending through a portion of the body 150 of the manifold 102, and a second channel 180 defined by an outer, distal surface of the manifold 102 and proximal surfaces of the valve seal 136 and valve seal seat 138. The channels 178 and 180 fluidly couple with each other to form a continuous conduit between the first chamber to the third chamber 116. In FIG. 1, the first channel 178 and the second channel 180 intersect at a right angle; however, it should be appreciated that the arrangement shown is merely illustrative, and that other arrangements of the flow-through passage 126 are also contemplated.

In some embodiments, a flow control feature 128 is formed within the flow-through passage 126 to facilitate controlled flow of fluids through the flow-through passage 126 and prevent excessive fluid flow. The flow control feature 128 may be referred to as a “flow choke” in some examples herein. In the embodiment of FIG. 1, the flow control feature 128 includes a series of bends in the first channel 178, and/or a portion of channel having reduced dimensions (e.g., a portion of first channel 178 having a reduced diameter to form flow control feature 128) to form a venturi-type valve; however, it should be appreciated that the position, type, and arrangement shown is merely illustrative, and that other positions, types, and/or arrangements of the flow control feature 128 are also contemplated. For example, other types of valves, including passive valves other than venturi-type valves, are contemplated.

The first diaphragm 118 and the second diaphragm 122 span between one or more inner surfaces of the manifold 102 to partially define one or more of the first chamber 112, the second chamber 114, and third chamber 116. Generally, the first diaphragm 118 is disposed proximal to the second diaphragm 122 and separates the first chamber 112 from the second chamber 114, creating a fluidic seal therebetween. In addition, the second diaphragm 122 separates the second chamber 114 from the third chamber 116 and creates a fluidic seal therebetween. Each of the first diaphragm 118 and the second diaphragm 122 includes a substantially planar membrane 170 or 172, respectively, formed of a flexible and elastic material that is configured to deflect upon application of force(s), such as pressure and/or contact forces, and rebound or return to a resting state upon withdrawal of such force(s). Examples of suitable flexible and elastic materials include silicone rubber, thermoplastic elastomers, or combinations thereof, and in durometers ranging from 30 to 90 on the Shore A hardness scale. The membranes 170 and 172 may be fixedly coupled to the manifold 102 via any suitable methods, such as ultrasonic welding or the like.

The first diaphragm 118 further includes, or is coupled to, a pin 120 on a distal side of the first diaphragm 118, while the second diaphragm 122 includes, or is coupled to, a pin 124 on a distal side of the second diaphragm 122. In some embodiments, the pin 120 and/or the pin 124 is integrally formed with the first diaphragm 118 or the second diaphragm 122, respectively. In such embodiments, the pin 120 and/or the pin 124 may be formed by a portion of the first diaphragm 118 or the second diaphragm 122, respectively, having greater thickness than the remainder of the first diaphragm 118 or the second diaphragm 122. For example, the pin 120 and/or the pin 124 may include a portion of the first diaphragm 118 or second diaphragm 122 having larger thickness as compared to the membrane 170 or membrane 172. Accordingly, the pin 120 and/or the pin 124 may be formed of the same material as the membrane 170 or membrane 172, respectively. In other embodiments, the pin 120 and/or the pin 124 are separate components fixedly coupled (for example, via ultrasonic welding or over-injection molding) to the membrane 170 or membrane 172, respectively. In such embodiments, the pin 120 and/or the pin 124 may be formed of a rigid material, such as a high density polyethylene (HDPE), a polypropylene (PP), a cyclic olefin copolymer (COC), a cyclic olefin polymer (COP), a polyetherimide (PEI), and combinations thereof.

In some embodiments, the pin 120 is configured to stay in contact against the second diaphragm 122, and/or the pin 124 is configured to stay in contact against the valve core 130, at all times (even in a resting state). In certain other embodiments, however, the pin 124 configured to contact the second diaphragm 122, and/or the pin 124 is configured to contact the valve core 130, only when the first diaphragm 118 and/or the second diaphragm 122 is deflected. During use, the pin 120 is configured to press against a proximal side of the second diaphragm 122 when the first diaphragm 118 is deflected distally by pressure forces from fluids in the first chamber 112. In turn, the contact by the pin 120 causes the second diaphragm 122 to deflect distally, thereby causing the pin 124 to press against the valve core 130 to displace the valve core 130 distally. This “chain reaction” facilitates the opening of a flow path between the first chamber 112 and the nozzle 160 for fluids to be dispensed from the dispensing device 100, as described in further detail below.

In the embodiment of FIG. 1, the membrane 170 of the first diaphragm 118, including pin 120, has a greater lateral surface area than the membrane 172 of the second diaphragm 122, including pin 124. In other words, the first diaphragm 118 and pin 120 have a wider total cross-section as compared to the second diaphragm 122 and pin 12. This enables any fluid pressure force(s) pressing distally against the first diaphragm 118 to supersede opposing proximal forces against the second diaphragm 112 during fluid dispensing, as discussed in more detail below. To facilitate the variance in surface areas, the first diaphragm 118 may span across a portion of the manifold 102 having a larger diameter, width, or other dimension as compared to the portion of the manifold 102 across which the second diaphragm 122 extends.

A vent port 140 extends through the body 150 of the manifold 102 and fluidically couples the first chamber 112 and the second chamber 114 to an environment external to the manifold 102. Generally, the vent port 140 includes one or more vent channels 142 and/or vent compartments 144 through which atmospheric gases (such as air) may pass into and/or out of the manifold 102.

In the embodiment of FIG. 1, the first chamber 112 is in fluid one-way communication with the external environment via a one-way valve 148 disposed at a proximal end of the single vent compartment 144, which has a microbial filter 146 arranged therein. During use, to facilitate pressure equalization in the dispensing device 100 (and the flexible container attached thereto), atmospheric gases from the external environment may pass through the one-way valve 148 in the vent port 140 to fill a volume in the first chamber 112 (and the flexible container) that was previously occupied by fluids that were expelled during dispensing. The one-way valve 148 is configured to tightly seal the vent compartment 144 when pressure within the dispensing device 100 (for example, pressure within the first chamber 112) and/or the flexible container attached to the dispensing device 100 is greater than external atmospheric pressure. This, in turn, prevents fluid(s) from leaking into the vent compartment 144 and contaminating, clogging, or otherwise fouling the microbial filter 146.

The microbial filter 146 is disposed in the vent compartment 144 to prevent microbes and other contaminants from entering the first chamber 112 and the flexible container while allowing atmospheric gases to pass therethrough. In some embodiments, the microbial filter 146 is hydrophobic. To further limit the risk of contamination, the microbial filter 146 may be disposed at a distance from an external end of the vent port 140 (for example, the end adjacent the external environment).

Still with reference to the embodiment of FIG. 1, the second chamber 114 is in fluid two-way communication with the environment external via at least one vent compartment 142 of the vent port 140. During use, atmospheric gases from the external environment may fill and/or be expelled from the second chamber 114 to facilitate pressure equalization in the dispensing device 100 (and a flexible container attached thereto) when the first diaphragm 118 is being deflected or returned to its resting state.

In some embodiments, the fairing 104 is coupled with the manifold 102 such that a slight space 182 is formed between the inner surface 186 of the body 158 of the fairing 104 at the proximal segment 106 and the outer surface 184 of the body 150 of the manifold 102. This space 182 may extend at least between the most proximal ends of the manifold 102 and/or fairing 104 to the vent port 140. Accordingly, the “external environment” to or from which atmospheric gases are vented by the vent port 140 may include the space 182. By having atmospheric gases pass through the space 182 before being vented into the dispensing device 100, the ingress of microbes and other contaminants into the dispensing device 100 may be significantly reduced. Accordingly, the arrangement of the space 182 causes the fairing 104 to function as vent cover and prevent direct ingress of microbes and other contaminants. In some embodiments, to facilitate increased or unhindered gas flow into/out of the dispensing device 100 (and a flexible container attached thereto) via the vent port 140, one or more channels, grooves, or other passageways may be formed into the outer surface 184 of the manifold 102 and/or the inner surface 186 of the fairing 104.

The valve seal seat 138 is disposed between the fairing 104 and the manifold 102, and is configured to support and orient the fairing 104 relative to the manifold 102. In some embodiments, the valve seal seat 138 functions as an intermediary coupling that holds the fairing 104 and the manifold 102 together. In some embodiments, the valve seal seat 138 is further configured to support and maintain a position of the valve seal 136 against the inner surface of the fairing 104 at the distal segment 108 to facilitate a stable fluid pathway directly proximal to the valve seal 136, even when the valve cone 130 is pressed against the valve seal 136, as described in further detail below. The valve seal seat 138 may be snap fit or press fit to the fairing 104, the manifold 102, and/or the valve seal 136 during assembly to hold the various components of the dispensing device 100 together. In the embodiment of FIG. 1, the valve seal seat 138 is annular in shape, though other shapes and arrangements are also contemplated.

The valve seal 136 is configured to engage with the valve core 130 at the distal segment 108 of the fairing 104 to open and close the fluid path extending between the first chamber 112 and the nozzle 160. In the embodiment of FIG. 1, the valve seal 136 includes an annular member that is press fit, or snap fit, with the fairing 104 to surround at least a portion of the valve core 130. In some embodiments, the valve seal seat 138 further supports or holds the valve seal 136 in place.

The valve core 130 sits within a core conduit 134 in the distal segment 108 through which the valve core 130 may axially translate (for example, proximally and distally). In some embodiments, the core conduit 134 is substantially cylindrical in shape. The biasing device 132 is arranged within, or adjacent to, the core conduit 134 to bias the valve core 130 proximally toward the valve seal 136 while allowing fluid within the core conduit 134 to bypass the biasing device 132. In some embodiments, to allow fluids to bypass the biasing device 132 within the core conduit 134, the biasing device 132 has a lateral diameter, width, or other dimension that is less than a lateral diameter, width, or other dimension of the core conduit 134 within, or near which, the biasing device 132 is arranged.

Generally, the biasing device 132 is configured to press the valve core 130 toward the valve seal 136 with such force(s) that a microbially-resistant seal is formed between the valve core 130 and the valve seal 136 when no opposing distal force(s) are present. Further, the amount of biasing force(s) provided by the biasing device 132 determines the amount of force(s) necessary to dispense fluids through/from the dispensing device 100. The biasing device 132 may include any suitable type of biasing device, such as a leaf spring, flat spring, disk spring, coil spring, and the like. In some embodiments, the biasing device 132 is further configured as a flow control device to control the flow of fluids through the core conduit 134, similar to flow control feature 128. For example, the biasing device 132 may be configured to reduce the occurrence of fluid streaming through the exit channel 110.

A proximal outer surface 176 of the valve core 130 and/or an inner surface 174 of the valve seal 136 are shaped and oriented to mate together and form a fluidic seal when the valve core 130 is pressed against the valve seal 136 by the biasing device 132. Accordingly, when the valve core 130 is pressed against the valve seal 136, the fluid path between the first chamber 112 and the nozzle 160 is closed. However, when the valve core 130 is displaced distally as caused by deflection of the second diaphragm 122, the fluid path is opened, and fluids may flow from the third chamber 116, into the core conduit 134, and out of an exit channel 110 of the nozzle 160.

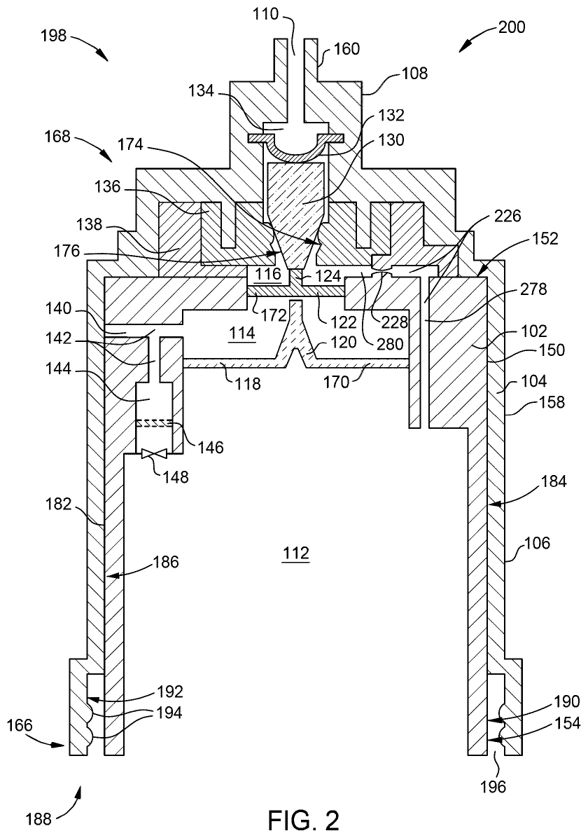

FIG. 2 is a schematic cross-sectional side view of a dispensing device 200 for dispensing fluids, according to some embodiments of the present disclosure. The dispensing device 200 is similar to the dispensing device 100 of FIG. 1. Accordingly, similar features are labeled with like reference numerals.

The dispensing device 200 differs from the dispensing device 100 in the arrangement of a flow-through passage 226. As shown in the embodiment of FIG. 2, the flow-through passage 226 comprises at least a first channel 278 extending through a portion of the body 150 of the manifold 102, and a second channel 280 defined by the outer, distal surface of the manifold 102 and the proximal surfaces of the valve seal 136 and valve seal seat 138. Similar to the arrangement in FIG. 1, the channels 278 and 280 fluidly couple with each other to form a continuous conduit between the first chamber to the third chamber 116. However, unlike the flow-through passage 126 of FIG. 1, the flow-through passage 226 of FIG. 2 includes a flow control feature 228 in the second channel 280 instead of the first channel 178/278. Further, the flow control feature 228 includes a portion of channel having reduced dimensions to form a venturi-type valve, without any bends in the second channel 278. Other features and/or arrangements may also prove equally effective. Thus, it should be appreciated that the position, type, and arrangement of the flow-through passage 226 as shown is merely illustrative, and that other positions, types, and/or arrangements are also contemplated.

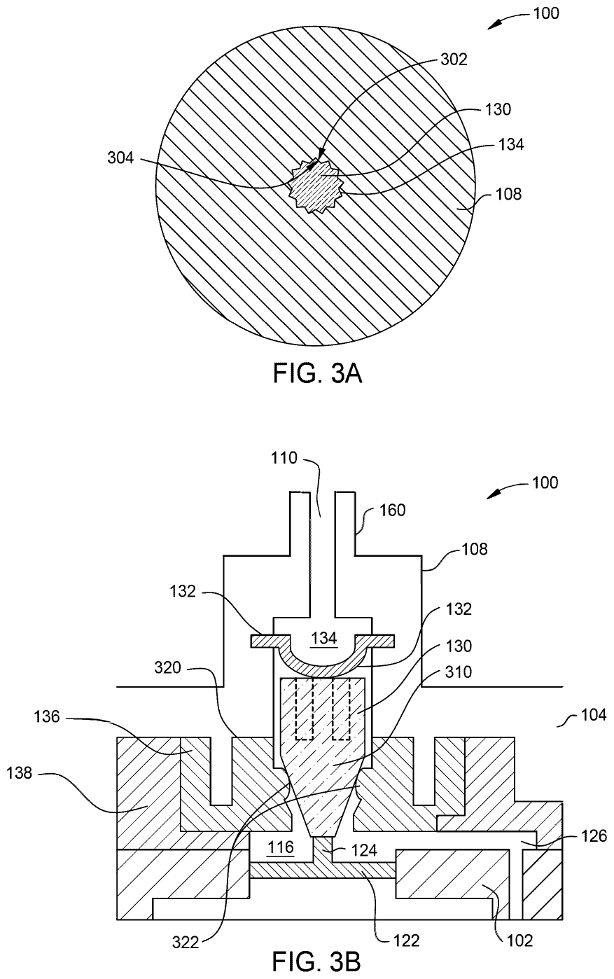

FIG. 3A is a partial schematic cross-sectional top view of the valve core 130 and the distal segment 108 of the fairing 104 of FIG. 1 or FIG. 2, according to some embodiments of the present disclosure.

As shown, at least a portion of the distal segment 108 of the fairing 104 forms the core conduit 134 through which the valve core 130 is disposed. During use, the valve core 130 may translate axially (for example, linearly) through the core conduit 134 as a result of biasing forces applied by the biasing device 132 and opposing pressure forces from fluids flowing distally in the first chamber 112, which are translated into deflections of the diaphragms 118 and 122 that ultimately press against the valve core 130.

To maintain a rotational and linear orientation of the valve core 130 within the core conduit 134 during translation thereof, an outer surface 302 of the valve core 130 and an inner surface 304 of the distal segment 108 surrounding the core conduit 134 may have reciprocal or nested cross-sectional shapes, and/or one or more corresponding mating features. For example, in the embodiment of FIG. 3A, the outer surface 302 of the valve core 130 and the inner surface 304 of the distal segment 108 have corresponding stellated tridecagon-shaped cross-sections, which prevent the valve core 130 from rotating or tilting in the core conduit 134. However, the illustrated cross-sections are merely illustrative, and other cross-sectional shapes are also contemplated. In some embodiments, the cross-sectional shape may extend along an entire axial length of the valve core 130 and/or the distal segment 108 forming the core conduit 134; in other embodiments, the cross-sectional shape extends along only a portion of the axial length of the valve core 130 and/or the distal segment 108 forming the core conduit 134. In further embodiments, the valve core 130 and/or the distal segment 108 include one or more corresponding mating features, such axial grooves, seams, protrusions, flanges, and the like, that allow the valve core 130 to translate axially through the core conduit 134 while maintaining a rotational and axial orientation of the valve core 130.

In some embodiments, the size and/or shape of the valve core 130 and/or the core conduit 134 can be selected based on a target downstream flow control parameter, such as fluid flow speed and fluid streaming. In some embodiments, the size and/or shape of the valve core 130 and/or the core conduit 134 can be selected based on a material of the valve core 130 and/or the portion of the fairing 104 forming the core conduit 134. In further embodiments, the size and/or shape of the valve core 130 and/or the core conduit 134 can be selected based on a desired coefficient of friction between the components.

FIG. 3B is a partial schematic cross-sectional side view of the dispensing device 100 illustrating at least the valve core 130, the biasing device 132, the valve seal 136, and the valve seal 138, according to some embodiments of the present disclosure.

As shown in FIG. 3B, the valve core 130 and the valve seal 136 may each include one or more tapered surfaces that allow the valve core 130 and the valve seal 136 to engage with each other to control and/or limit translation of the valve core 130 within the core conduit 134, and to form a fluidic seal therebetween. For example, in the embodiment of FIG. 3B, a proximal segment 310 of the valve core 130 is tapered, or conical, in shape. Similarly, a medial portion 320 of the valve seal 136 has a corresponding inward taper. The tapered proximal segment 310 of the valve core 130 is configured to nest with the tapered medial portion 320 of the valve seal 136 as the valve core 130 is pressed against the valve seal 136 by the biasing device 132. The size and/or shape of the taper in the proximal segment 310 and/or medial portion 320 may be selected to prevent the valve core 130 from being proximally translated into the valve seal 136 beyond a certain distance as a result of the valve core 130 being actuated during fluid dispensing.

Generally, the interface between the valve core 130 and the valve seal 136 is the primary fluidic seal for the fluid path within the dispensing device 100. Accordingly, a material and/or surface finish of the valve core 130 and/or the valve seal 136, such as the proximal segment 310 and medial portion 320, may be selected to optimize the sealing characteristics of the valve core 130 and the valve seal 136. In some embodiments, the valve core 130 is formed of a rigid and hydrophobic material, while the valve seal 136 is formed of an elastic and hydrophobic material. Examples of suitable rigid materials include polyethylene such as a low-density polyethylene (LDPE), a high-density polyethylene (HDPE), a cyclic olefin copolymer (COC), a polyetherimide (PEI), a polytetrafluoroethylene (PTFE), a polyoxymethylene (POM), and combinations thereof. Examples of suitable elastic materials may include silicone or thermoplastic elastomers. In some embodiments, the elastic material of the valve seal 136 is selected based on a hardness value of the material to provide appropriate sealing force(s) or pressure against the valve core 130. For example, in some embodiments, the elastic material has a Shore A hardness value between about 30 and about 80. In further embodiments, the valve core 130 is instead formed of the elastic hydrophobic material, while the valve seal 136 is formed of the rigid and hydrophobic material. In still further embodiments, both of the valve core 130 and the valve seal 136 are formed of a rigid material or an elastic material. Generally, each of the valve core 130 and the valve seal 136 have a highly polished surface finish, particularly at the proximal segment 310 and medial portion 320.

In the embodiment shown in FIG. 3B, the valve seal 136 further includes one or more sealing features 322 extending inwardly from the medial portion 320. The sealing features 322 include projections configured to contact and press against the proximal segment 310 of the valve core 130 to form an enhanced fluidic seal. Generally, the sealing features 322 can have any suitable shape and/or size depending on a shape of the valve core 130 and the desired performance characteristics of the valve, including sealing characteristics of the valve seal 136 and/or the amount of requisite force(s) for pushing the valve core 130 distally and dispensing fluids. Examples of suitable shapes for the sealing features 322 include elliptical, hemispherical, triangular, trapezoidal, or similar shapes, including shapes with angles not normal to the surrounding valve seal 136. In some embodiments, the one or more sealing features 322 include a single protrusion extending from and circumscribing an inner surface of the medial portion 320. In some embodiments, the one or more sealing features 322 include a plurality of protrusions extending from and annularly arranged on an inner surface of the medial portion 320.

In some embodiments, the valve seal 136 has a substantially uniform cross-section around the circumference of the valve core 130. In some embodiments, however, the valve seal 136 may have a non-uniform cross-section around the circumference of the valve core 130. For example, in such embodiments, a thickness of the valve seal 136 and/or the size of one or more sealing features 322 may be reduced in one or more select areas around the circumference of the valve core 130. Alternatively or in addition thereto, a shape of the valve seal 136 or the one or more sealing features 322 in the select area(s) can be varied. Such reduction in thickness or size, or change in shape, may reduce the requisite actuation force(s) for fluid(s) to bypass the valve core 130 in such areas, which can improve overall control of streaming as the fluid(s) exit the interface between the valve core 130 and valve seal 136. In some embodiments, the shape of the valve seal 136 or the one or more sealing features 322 can be varied in the select area(s) to increase the requisite actuation force(s) for fluid(s) to bypass the valve core 130 in such areas, which can also provide a similar effect on fluid flow control.

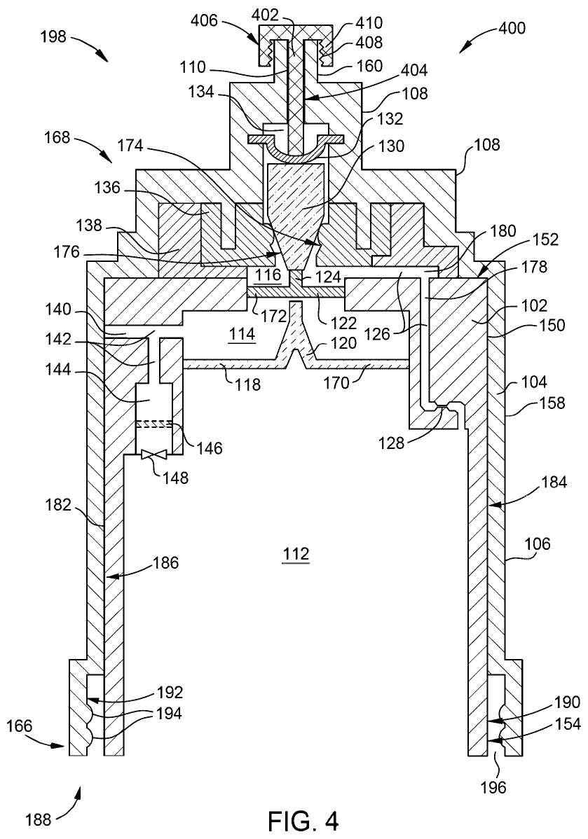

FIG. 4 is a schematic cross-sectional side view of a dispensing device 400 with a cap 402 disposed thereon, according to some embodiments of the present disclosure. The dispensing device 400 is similar to the dispensing devices 100 and 200 of FIGS. 1 and 2, respectively. Accordingly, similar features are labeled with like reference numerals.

In the embodiment of FIG. 4, the dispensing device 400 is configured to be sealed by the cap 402 for storage (for example, between uses, or for long term storage). The cap 402 removably attaches to the nozzle 160 to seal off, or block, a distal end of the exit channel 110 from the external environment, thereby obstructing or preventing microbes or other contaminants from entering the dispensing device 400 during storage, as well as preventing fluids form unintentionally spilling out of dispensing device 400. In some embodiments, the cap 402 is configured to secure onto the nozzle 160 via a threaded connection. In such embodiments, a top portion 406 of the cap 402 may include one or more threads or grooves 408 disposed on an inner surface thereof and configured to mate with one or more threads or grooves (not shown) disposed on an outer surface of the nozzle 160. In other embodiments, however, the cap 402 can be secured onto the nozzle 160 via a snap fit, friction fit, clasp, or the like.

In some embodiments, the cap 402 includes a plug 404 attached to, and extending from, the top portion 406. As shown in FIG. 4, the plug 404 is configured to slide into the exit channel 110 and function as a stopper to further prevent contaminants from entering the dispensing device 400, and to prevent fluids from unintentionally spilling out. Further, the plug 404 may function to reduce the volume of fluid contained in the exit channel 110 and/or the core conduit 134 during storage, which reduced the chances of microbial growth in these passages that can contaminate a next dispensed dose of fluid(s). Generally, the plug 404 may include an elongated body shaped and sized to substantially occupy, or fill, the exit channel 110. In some embodiments, the plug 404 may be sized to extend to, and in some embodiments contact, the biasing device 132 when the cap 402 is secured onto the dispensing device 400. In such embodiments, the plug 404 may function to inhibit distal translation of the valve core 130 by preventing distal deflection or movement of the biasing device 132 pressing against the valve core 130. This prevents accidental movements of the valve core 130 as caused by, for example, unintended and/or rapid movements of a flexible container to which the dispensing device 400 is attached, which may lead to fluid leakage and/or microbial ingress. Further, in some scenarios, it may be beneficial to have the fluid path of the dispensing device 400 remain closed, even when the pressure in the chamber 112 increase to a point that would normally cause the fluid path to open.

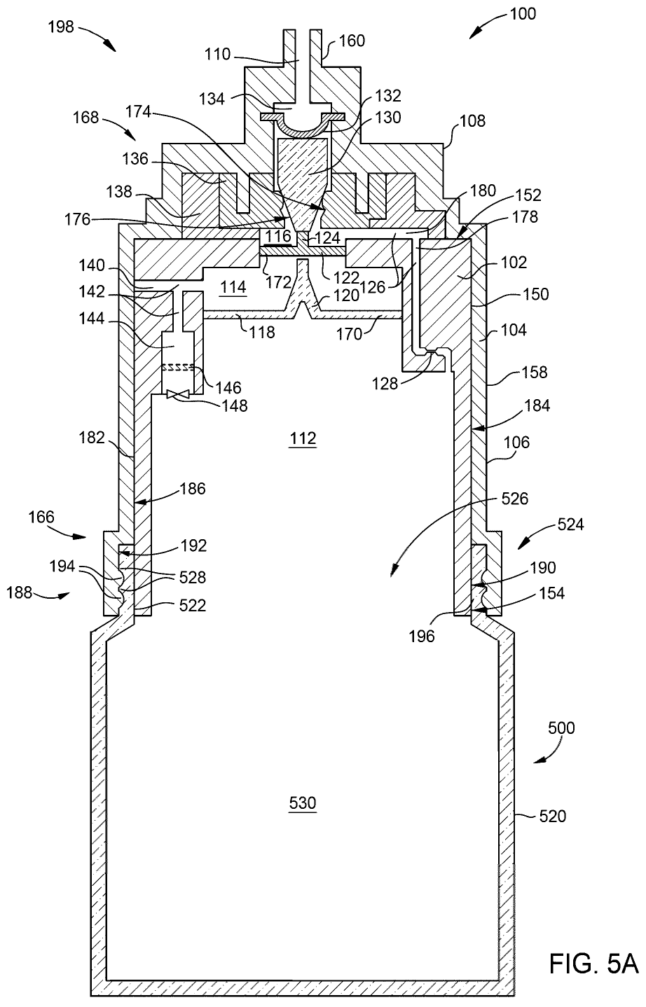

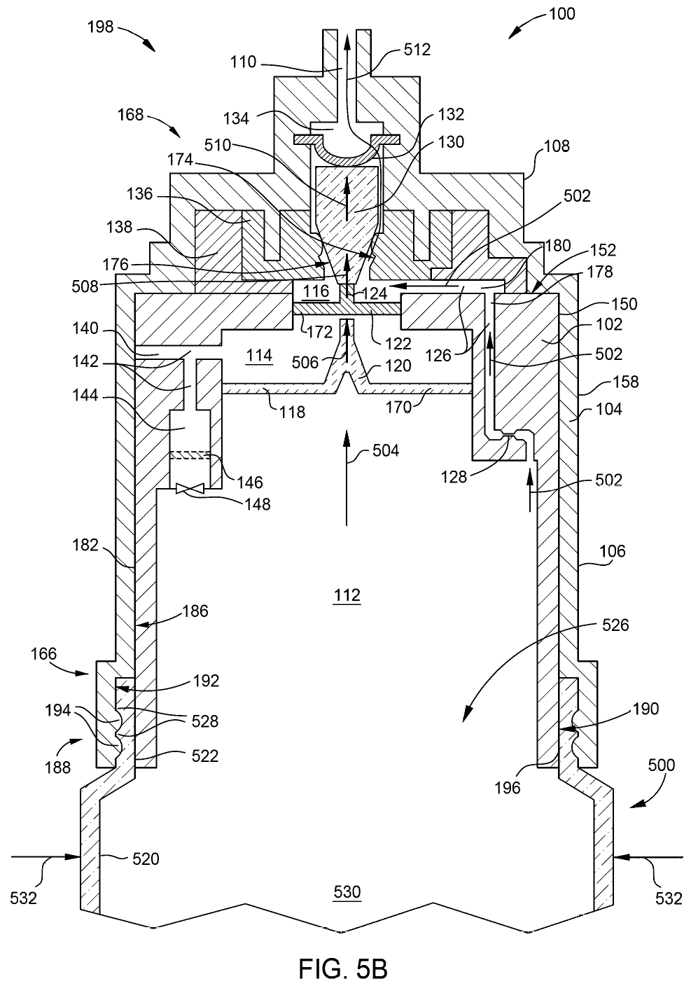

FIG. 5A is a schematic cross-sectional side view of dispensing device 100 when attached to a flexible container 500, according to some embodiments of the present disclosure. Meanwhile, FIG. 5B is a schematic cross-sectional side view of the dispensing device 100 and the flexible container 500 during dispensing, with arrows indicating movement of components and flow paths of fluid(s) in the dispensing device 100, according to some embodiments of the present disclosure. For clarity, FIGS. 5A and 5B are described together herein. Further, it should be appreciated that FIGS. 5A and 5B are merely illustrative, and that the methods illustrated in FIGS. 5A and 5B may also be applied to other dispensing devices described herein having other arrangements.

Generally, the flexible container 500 is representative of any suitable type of container for storing and dispensing ophthalmic fluids, including, without limitation, an eye drop dispenser, droptainer, drop bottle, squeeze bottle, dropping bottle, squeeze tube, and the like. Examples of ophthalmic fluids that can be stored and dispensed from the flexible container 500 include, without limitation, lubricating eye drops, medicated/prescription eye drops, ophthalmic gels, ophthalmic ointments, ophthalmic stains, other preservative free ocular solutions, and the like.

As shown in FIGS. 5A and 5B, the flexible container 500 includes a body 520 defining a volume 530. The body 520 further includes a neck 522 at a dispensing end 524 that at least partially defines an opening 526 through which fluids may be dispensed from or loaded into the volume 530. To assemble the dispensing device 100 and the flexible container 500, the base 188 of dispensing device 100 can be fixedly or removably attached to the neck 522 such that the dispensing device 100 covers or caps the opening 526, and any fluids exiting the volume 530 of the flexible container 500 are received in the first chamber 112 of the dispensing device 100. Accordingly, when assembled, the first chamber 112 of the dispensing device 100 is open to, and in fluid communication with, the volume 530 of the flexible container 500.

In some embodiments, the neck 522 is received and secured in the annular gap 196 of the dispensing device 100 during attachment. In such embodiments, the portion 192 of the inner surface 186 of the fairing 104, and/or the portion 190 of the outer surface 184 of the manifold 102, may have feature(s) 194 configured to mate with corresponding feature(s) 528 on an outer surface of the neck 522 to secure or fasten the dispensing device 100 to the flexible container 500. In some embodiments, the feature(s) 194 and corresponding feature(s) 528 may include complementary threads, protrusions, grooves, or combinations thereof. Accordingly, the dispensing device 100 may be screwed, snap fit, press fit, friction fit, etc., onto the flexible container 500 during assembly.

With reference to FIGS. 5A and 5B collectively, to actuate the dispensing of fluid(s) through the dispensing device 100, a user (or other actuation means) may compress the body 520 of the flexible container 500. For reference, compression of the flexible container 500 is represented by the arrows 532 in FIG. 5B. The compression of the flexible container 500 causes pressure of fluid(s) within the volume 530 and the first chamber 112 in fluid communication with the volume 530 to increase, and a flow of fluid(s) from the volume 530, through the first chamber 112, through the flow-through passage 126, and into the third chamber 116. The flow of fluid(s) into the third chamber 116, represented by arrows 502 in FIG. 5B, results in increased fluid pressure within the third chamber 116, which imparts force(s) upon a distal surface of the second diaphragm 122 to displace the second diaphragm 122 proximally.

The increase in fluid pressure in the first chamber 112 also imparts force(s) on a proximal surface of the first diaphragm 118 to displace the first diaphragm 118 distally, as represented by the arrow 504. Since the surface area of the first diaphragm 118 is larger than a surface area of the second diaphragm 122, the distal displacement of the first diaphragm 118 overcomes the proximal displacement of the second diaphragm 122, and the pin 120 of the first diaphragm 118 contacts and pushes the second diaphragm 122 distally, as represented by the arrow 506. Eventually, as the amount of force(s) imparted by the fluid pressure in the first chamber 112 increases, the pin 124 of the second diaphragm 122 contacts and presses distally against the valve core 130, as represented by the arrow 508.

In a resting state, the valve core 130 is pressed proximally against the valve seal 136 by the biasing device 132. However, as the fluid pressure in the first chamber 112 increases, the distal force(s) imparted on the first diaphragm 118 and the second diaphragm 122 supersede the biasing forces of the biasing device 132 and cause the pin 124 to push the valve core 130 distally away from the valve seal 136, as represented by arrow 510. In this position, the 130 may be described as being “unnested” from the valve seal 136. This opens the fluid path between the first chamber 112 and the exit channel 110, and enables fluid(s) in the third chamber 116 to flow past (for example, bypass) the valve seal 136 and the valve core 130 and into the core conduit 134 and exit channel 110, as represented by arrow 512. The speed of fluid flow along the fluid path is moderated in some embodiments by the flow control feature formed within the flow-through passage 126. Under pressure, the flow of fluid(s) continues past the valve core 130 until there is sufficient volume of fluid(s) at the distal end of the nozzle 160 that a drop separates under, for example, the force of gravity, and is dispensed.

When the compressive forces 532 against the flexible container 500 are reduced or released, or the fluid pressure within the flexible container 500 and dispensing device 100 drops by other means, the biasing device 132 presses the valve core 130 back against the valve seal 136, closing the fluid path between the first chamber 112 and the exit channel 110 and preventing any backflow of the fluid(s). The drop in pressure also causes the first diaphragm 118 and the second diaphragm 122 to return to their respective, resting positions. In some embodiments, the drop in pressure causes proximal displacement of the first diaphragm 118 and the second diaphragm 122 beyond their resting positions, as the fluid volume in the first chamber 112 and volume 530 is smaller after dispensing.

Still with reference to FIGS. 5A and 5B collectively, negative pressure in the first chamber 112 and volume 530 after the reduction or release of the compressive forces 532 is equilibrated by gas flow through the vent port 140 and into the first chamber 112 and volume 530. The microbial filter 146 and one-way valve 148 facilitate the return of sterile atmospheric gases (for example, sterile air) into the dispensing device 100 and flexible container 500 to balance internal and external pressures and allow the dispensing device 100 and flexible container 500 to expand/relax.

Generally, the various components of the dispensing devices 100, 200, and/or 400 can be individually fabricated by any suitable molding techniques, such as by injection molding and/or liquid silicone rubber injection molding, and/or three-dimensional (3D) printing techniques, and thereafter press fit, snap fit, friction fit, or otherwise coupled together during assembly. In some embodiments, however, one or more components of the dispensing device 100 can be monolithically molded and/or 3D printed. Similarly, one or more of the components of the flexible container 500 can be fabricated by molding techniques, such as by injection molding and/or liquid silicone rubber injection molding, and/or 3D printing techniques, and thereafter press fit, snap fit, friction fit, or otherwise coupled together during assembly. In some embodiments, however, the flexible container 500 is monolithically molded and/or 3D printed.

In summary, embodiments of the present disclosure provide an apparatus for dispensing fluids (including, without limitation, liquids, gels, solutions, emulsions, suspensions, and the like) from flexible containers, while providing improved fluid flow control and prevention of microbial ingress into the containers. In some embodiments, the apparatuses of the present disclosure include a multistage valve mechanism that prevents microbial ingress when dispensing fluids from the aforementioned flexible containers, while still allowing passive ingress of air. The valve mechanism also facilitates the reduction of pressure for actuating the valve mechanism by a user, regardless of the viscosity of the solution being dispensed. Accordingly, embodiments of the present disclosure relate to a valve mechanism that effectively resists microbial contamination, allows passive air entry, and reduces the force(s) for operating and/or controlling the speed of fluid flow, thereby facilitating improved fluid preservation and flow control.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more. ” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S. C. § 112(f) unless the element is expressly recited in the claims using the phrase “means for” or, in the case of a method claim, the element is recited using the phrase “step for.”

While various examples have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described examples.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘including’ as used herein is synonymous with ‘including,’ ‘containing,’ or ‘comprising,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the described subject matter, but instead as merely intended to highlight alternative or additional features that may or may not be utilized in a particular example of the described subject matter. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the described subject matter to the specific examples and examples described herein, but rather to also cover all modification and alternatives coming with the true scope and spirit of the described subject matter.

What is claimed is:

1. An apparatus for dispensing fluids, comprising:
a manifold;
a fluid path at least partially disposed in the manifold, the fluid path comprising an inlet and an outlet; and
a multistage valve at least partially disposed in the manifold, the multistage valve comprising:
a core positioned in the fluid path between the inlet and the outlet;
a seal positioned around the fluid path between the inlet and the outlet, the core nested with the seal;
a biasing device oriented to cause a first force to be exerted on the core, the first force biasing the core in a first direction against the seal to close the fluid path; and
a plurality of diaphragms positioned adjacent to the fluid path, wherein the plurality of diaphragms is configured to act upon the core in response to fluid pressure on an inlet side of the fluid path exerting a second force against the plurality of diaphragms to at least partially unnest the core from the seal.

2. The apparatus of claim 1, wherein:
the second force biases the plurality of diaphragms in a second direction opposite the first direction to at least partially unnest the core from the seal to open the fluid path when the second force supersedes the first force.

3. The apparatus of claim 2, wherein the plurality of diaphragms comprises:
a first diaphragm comprising a first pin, the first diaphragm disposed adjacent to and at least partially defining a first chamber on the inlet side of the fluid path and a second chamber disposed between the inlet side of the fluid path and an outlet side of the fluid path; and
a second diaphragm comprising a second pin, the second diaphragm disposed adjacent to and at least partially defining the second chamber and a third chamber disposed between the inlet side of the fluid path and an outlet side of the fluid path.

4. The apparatus of claim 3, wherein the second force biases the first diaphragm toward the second diaphragm to cause the first pin to press against the second diaphragm.

5. The apparatus of claim 4, wherein the first pin pressing against the second diaphragm causes the second pin to press against the core to open the fluid path.

6. The apparatus of claim 3, further comprising:
a vent path disposed between the first chamber and an external environment;
a filter disposed in the vent path; and
a one-way valve oriented to allow fluid to passively enter the first chamber from the vent path.

7. The apparatus of claim 6, wherein fluid is vented into the first chamber during return of the plurality of diaphragms to a resting position from a biased position.

8. The apparatus of claim 6, wherein the filter comprises a hydrophobic filter.

9. The apparatus of claim 6, further comprising:
a fairing at least partially disposed around the manifold, wherein the external environment comprises a space disposed between an inner surface of the fairing and an outer surface of the manifold.

10. The apparatus of claim 2, further comprising:
a fairing at least partially disposed around the manifold, the fairing forming at least a portion of an outlet side of the fluid path.

11. The apparatus of claim 10, wherein the fairing forms a nozzle at the outlet side of the fluid path.

12. The apparatus of claim 11, further comprising:
a cap configured to removably couple to the fairing at the outlet side of the fluid path to seal the fluid path.

13. The apparatus of claim 12, wherein the cap is further configured to act against the biasing device when coupled to the fairing to prevent the core from separating from the seal.

14. The apparatus of claim 10, wherein the manifold and the fairing are configured to attach to a flexible container.

15. The apparatus of claim 14, wherein the flexible container comprises a squeeze tube or squeeze bottle.
